(19) United States
(12) Patent Application Publication
(10) Pub. No.: US 2005/0089943 A1
(43) Pub. Date: Apr. 28, 2005

(54) Rapid method of determining clearance of prion protein

(76) Inventors: Kang Cai, Chapel Hill, NC (US); Christopher J. Stenland, Cary, NC (US)

Correspondence Address: WOMBLE CARLYLE SANDRIDGE & RICE, PLLC, P.O. BOX 7037, ATLANTA, GA, 30357-0037, US

(21) Appl. No.: 10/693,734

(22) Filed: Oct. 23, 2003

(51) Int. Cl. G01N 33/53; G01N 33/537; G01N 33/543

(52) U.S. Cl. 435/7.92

(57) ABSTRACT

The invention provides a rapid, sensitive immunoassay capable of detecting and quantitating pathogenic protein to a level of 3 to 5 logs. The preferred immunoassay utilized is a chemiluminescent endpoint for a Western blot immunoassay. The invention has been successfully applied to track the clearance of pathogenic protein during production of proteins derived from plasma. It is particularly applicable and has been confirmed by bioassay to relate TSE infectivity to quantitative results on prion protein.

### FIELD OF THE INVENTION

The invention relates to a rapid method of detecting pathogen or prion protein that may be used to determine the clearance of pathogen protein, in general, and to a Western blot immunoassay method of relating pathogen protein clearance to infectivity clearance, in specific. The method has been applied to the quantitation of TSE protein clearance and its relationship to infectivity clearance.

### BACKGROUND OF THE INVENTION

The Cohn-Oncley purification of therapeutic proteins from blood plasma, referred to herein in general as the Cohn process or scheme, employs a series ethanol additions and pH adjustments to purify or enrich for proteins which may be used in human therapies. Commonly purified proteins include immunoglobulins, anti-hemophiliac factors and albumin. While many manufacturers of such products utilize the basic Cohn scheme, frequently established steps may be modified or additional steps are implemented to increase either the purity and/or yield for a given product. Such steps are typically proprietary for a given manufacturer.

Since the discovery that HIV could be carried and transmitted through the use of blood products, the interest and concern about the presence of such pathogenic agents in biological products derived from blood has increased. Most recently, there has been concern that CJD, Creutzfeldt-Jakob Disease, could be transmitted through the use of blood-derived products. CJD is one of the human transmissible spongiform encephalopathies (TSE), a collection of neurodegenerative diseases that are debilitating and fatal. Infectivity associated with CJD appears to be either associated with or caused by the prion protein (PrP). Although new disease carrying viruses may be generated at any time, manufacturers of blood-based products take precautions to obtain a blood product that is free of known transmissible diseases, to the extent for which these can be tested. Unfortunately, the primary test for possible TSE infectivity is a biological assay in which rodents are injected with the material of interest to see if infectivity develops. The results of such assays require nine months to a year to develop, frequently too long to hold a manufactured lot of plasma product prior to release for use.

Therefore, a method of detecting a protein associated with a pathogen suspected of carrying infectivity such as the prion or viral surface (coat) protein is important for the blood fractionation industry. A rapid, sensitive method capable of determining the removal of virus or pathogenic prion protein would provide the blood fractionation industry with a useful tool for determining what danger of infectivity exists after a particular manufacturing process step. The decrease in viral or prion protein relative to a given product associated with a manufacturing process step is referred to herein as “clearance”. Because of the importance of such a test for TSE infectivity to the safety of plasma products, the method of this invention was described generally at the Blood Safety and Screening conference held in McClean, Va. on Feb. 23, 1998.

### SUMMARY OF THE INVENTION

The invention is an immunoassay method of detecting viruses or prion protein content of a biological sample. This method provides a quantitative measure of the viral or prion protein content that may be related to infectivity. The method can typically detect a range of prion protein from 3 logs to 5 logs dynamic range, and the measured clearance correlates well with infectivity clearance for the process steps have been tested. The preferred immunoassay method is a Western blot and results are available in 2-4 days. The method is particularly useful to track the prion protein related to potential infectivity in plasma production.

The method of the invention is composed of the steps of preparing: a) a biological sample, usually a plasma or plasma manufacturing intermediate sample, for an immunoassay, either a Western blot immunoassay or an ELISA immunoassay; b) performing the immunoassay for the protein associated with infectivity: c) quantitating the protein results; and, d) relating the protein results to infectivity. Preferably the quantitative method employs a Western blot immunoassay method. This method may be used to determine clearance of the pathogenic protein in a biological sample such as a plasma product or plasma processing sample by preparing an aliquot of a first sample; performing the Western blot assay on such first sample; quantitating the pathogenic protein results in the first sample; processing the first sample to obtain a second sample or samples from the process stream of the first sample; performing and quantitating the pathogenic protein results from the second sample or samples and comparing relative amounts of pathogenic protein detected in the first and second sample to determine the clearance of the processing step.

An application of particular interest is determining the clearance of TSE by a particular plasma processing step. The comparative quantitative results of the two immunoassays provide a measure of the “clearance” obtained by the processing step.

The preferred method is composed of the following steps:
a. spiking a process solution with a brain homogenate from an animal infected with the pathogen marker (typically a protein) of interest;
b. processing the spiked solution; and
c. assaying for the presence of the pathogen protein marker (prion protein) in the resulting fractions for distribution of the protein.

The assay step is composed of the following steps:
a. taking a sample of each fraction of interest;
b. diluting the samples in defined increments;
c. treating each diluted sample with proteinase-K;
d. (optionally) centrifuging the proteinase-K treated samples; and
e. performing a Western blot or ELISA immunoassay.

The preferred Western blot immunoassay is composed of the following steps:
f. separating the proteinase K treated samples electrophoretically;
g. transferring the separated samples to a membrane;
h. adding a blocking agent to the membrane containing the separated samples;
i. incubating the membranes with a first antibody capable of binding the pathogenic protein;
j. washing the incubated membrane with a low salt buffer to remove any non-binding antibodies and proteins;
k. incubating the washed membrane with a second antibody capable of recognizing the first antibody, which second antibody contains a reporter group capable of providing a measurable signal; and
l. measuring the signal produced by counting the number of lanes with detectable signal.

The number of lanes with detectable protein from sample diluted in defined increments allows for the estimation of infectivity clearance for a sample when compared to the spiked input material (prove). For the TSE protein assay, a preferred first antibody is the monoclonal antibody, 3F4. However, the TSE-specific antibody used can be another TSE-specific antibody with demonstrated effectiveness according to the methods of the invention, including monoclonal, polyclonal, or recombinantly engineered antibodies or antibody fragments. These antibodies can be conformational-specific or specific for linear epitopes.

### DESCRIPTION OF THE INVENTION

Determining the risk of transmission by blood or plasma-derived products of an infective virus or prion protein requires sensitive and specific assays for the detection of either infectivity or a reasonable marker for infectivity. This invention provides an immunoassay that fulfills all criteria: sensitive, specific, fast and low cost. Described in detail herein is one application of the invention, a Western blot immunoassay, that is both sensitive and reproducible for the detection of PrP^{RES}, a marker for transmissible spongiform encephalopathy (TSE) infectivity. One of skill in the art of such assays will be able to apply the method provided to the determination of the risk of transmission of other types of infectivity.

The method of the invention utilizes an immunologically-based assay, such as a Western blot or an ELISA technique, to monitor for the presence of the pathogenic form of the prion protein through a manufacturing process of a plasma- or biologically-derived product. The preferred method of the invention utilizes a sensitive Western blot immunoassay to detect the pathogenic form of PrP (PrP^{Sc}), referred to herein at times as TSE protein, in a series of carefully made dilutions made from samples containing an unknown amount of PrP. The invention involves spiking of a plasma process solution with the brain homogenate from an infected animal (such as hamster, mouse, sheep or human) that contains the pathogenic prion protein (PrP^{Sc}). An aliquot is removed for analysis (prove sample) and the remaining material is processed. Upon completion of the process step, the resulting fractions, typically an effluent (or liquid fraction) and a precipitate (or solid fraction), are obtained. The solid fraction is resuspended in an appropriate buffer such as phosphate buffered saline (PBS) or tris buffered saline (TBS) to the same volume as the other samples. All samples (prove, effluent and precipitate) are diluted in defined increments (e.g. 0.5 logs or 1 log steps) and each are treated with proteinase K to remove the non-pathogenic prion (PrP^{C}) and other proteins that may cross-react and interfere with the PrP signal. In some cases these proteinase-K treated dilations are centrifuged and the resulting pellet contains more concentrated PrP. If the Western blot is used, the samples to be analyzed are electrophoretically separated and then transferred to a membrane (such as nitrocellulose or PVDF). These membranes are blocked in the presence of (a) blocking agent(s) which may include but not be limited to, non-fat dried milk, serum albumin or casein. The membranes are incubated in the presence of either a monoclonal or polyclonal antibody, the “first antibody”, then washed with a low salt buffer to remove non-binding antibodies and proteins. The washed membranes are then incubated with a second antibody or antibodies that recognize the first antibody. The second antibody has a reporter group attached for the visualization of the first antibody. This reporter that is attached to the second antibody may be either a chemical “tag” or an enzyme (such as alkaline phosphatase, horseradish peroxidase) that catalyzes a reaction that converts an added substrate to a product that is visualized. The method of visualization may include chemiluminescent, fluorescence or calorimetric technologies. For the specific TSE assay given herein the preferred visualization method is chemiluminesent and based on a alkaline phosphatase reporter conjugated to the second antibody. If the assay employed is similar to an ELISA, the electrophoretic separation and membrane transfer is omitted and the assay is performed in a ELISA plate. Quantitation of the clearance assay employs a quantal format which compares the resulting positive lanes (Western blot) or wells (ELISA) detected for the input (prove) sample with one or more of the output fractions (e.g. paste and/or effluent).

This method differs from prior art in that it does not use the rodent bioassay to follow infectivity partitioning in process steps (Brown et al., Transfusion, Vol. 38, September 1998). By detecting the pathogenic subform of the prion protein, PrP^{Sc}, rather than monitoring for infectivity, the method saves a significant amount of time and money.

The method of the invention is exemplified in detail below for the important TSE assay. The assay is based on the identification of a marker protein, PrP^{Sc}, with the disease infectivity and the use of an antibody to that prion protein. The specific primary antibody used herein is the monoclonal antibody designated 3F4 which may be obtained from Dr. Richard Rubenstein at the New York Institute for Basic Research in Developmental Disabilities, Staten Island, N.Y. or from Chemicon, Temecula, Calif.

As an alternative to use of a primary antibody such as 3F4 in combination with a secondary antibody specific for the primary antibody, the primary antibody or antibody fragment may be directly labeled in order to facilitate TSE detection and/or quantitation without use of a secondary antibody. In some circumstances, e.g., where a substantial non-specific signal is generated by immunoglobulin, immunoglobulin fragments, or other non-specific proteins in the samples exposed to the secondary antibody, the use of a directly labeled primary antibody can result in substantial reduction of the non-specific signal. Unless specifically indicated to the contrary, “labeled antibody,” as used herein, means that a detectable label is associated with the subject antibody and that no labeled secondary antibody is necessary for detection of the target.

Accordingly, the present invention also includes alternative embodiments wherein the primary antibody, such as 3F4, or a fragment thereof, is directly labeled or conjugated using direct linkage to a signal-generating substance (label). Directly labeled antibodies can be prepared by attachment of labels such as radio labels, enzyme labels, co-factor labels, fluorescent labels, paramagnetic labels, chemiluminescent labels, or metal labels. Enzyme labels include, but are not limited to, alkaline phosphatase, horseradish peroxidase, amylate dehydrogenase, staphylococcal nuclease, δ-5-steroidisomerase, yeast alcohol dehydrogenase, α-glycerophosphate dehydrogenase, triose phosphate isomerase, ribonuclease, urease, catalase, glucose-6-phosphate dehydrogenase, glucoamylase, β-galactosidase, acetylcholinesterase, asparaginase, and glucose oxidase. Radio labels include, but are not limited to, radioisotopes such as iodine (¹²⁵I, ¹²¹I), carbon (¹⁴C), sulfur (³⁵S), tritium (³H), indium (¹¹²In), and technetium (⁹⁹Tc-m). Fluorescent labels include, but are not limited to, coumarin, cyanine, fluorescein, indocarbocyanine, tetramethyl Rhodamine, Rhodamine Red-X, Texas Red, and indodicarbocyanine. Alternatively, antibodies can be derivatized with a moiety that is recognized by a separately-added label, for example, biotin.

Techniques for chemically modifying antibodies with such labels are well-known in the art. Antibodies can also be conjugated to fluorophores, other proteins, and other macromolecules, using bifunctional linking reagents. Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available from Pierce, Rockford, Ill., USA).

Additionally, a wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other. Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X. A wide variety of other amine-reactive and thiol-reactive fluorophores are available commercially (Molecular Probes, Inc., Eugene, Oreg., USA), including Alexa Fluor.RTM. 350, Alexa Fluor.RTM. 488, Alexa Fluor.RTM. 532, Alexa Fluor.RTM. 546, Alexa Fluor.RTM. 568, Alexa Fluor.RTM. 594, Alexa Fluor.RTM. 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

Directly labeling of the TSE-specific antibody or antibody fragment, according to one aspect of the invention, allows the methods disclosed herein to be carried out without the use of a secondary antibody that may reactive with immunoglobulin, immunoglobulin fragments, or other non-specific proteins that may be present in particular samples. Those of skill in the art will recognize that various methods and substances can be used to conjugate or derivative antibodies in order to provide a detectable signal. The examples disclosed herein are provided for purposes of illustration and are not intended as limiting in any respect.

To assess the risk of TSE disease transmission in manufacturing processes for biological products, a sensitive in vitro assay was used as a predictive tool for tracking infectivity in plasma purification processes. The method of this invention is a sensitive, robust Western blot assay which is specific detection of PrP^{RES} (the proteolytically derived “core” or PrP^{Sc}) and therefore may be used to monitor the partitioning of PrP^{Sc}. The method of the invention has been successfully used to monitor the partitioning of PrP^{Sc} through three plasma protein purification steps critical to the isolation of various plasma therapeutics.

A major advantage of the Western blot format rather than other in vitro formats is the electrophoretic separation step that precedes immunologic detection. The Western blot image for PrP^{RES} demonstrates a distinct “fingerprint” of three protein bands of molecular masses ranging from 20 kD to 30 kD. The detection of these bands serves as visual confirmation that the signal observed is derived from PrP^{RES}. In addition to this protein “fingerprint”, the Western blot employs both antibody specificity and proteinase-K (PK) resistance of PrP^{Sc} to further insure assay reliability.

The Western blot assay of this invention is more sensitive than other assays described in the literature including dot blot, various ELISA systems, as well as other Western blot systems. In 1990 a dot blot assay format was used to detect PrP^{Sc}, but only with qualitative results. The dot blot assay, however, did not use the sensitive chemiluminescent detection system employed in this invention. As presented here, PrP^{Sc} may be detected in as little as 10 nanograms (ng) of brain equivalents. Other groups have reported Western blot detection systems for detecting PrP in brain homogenates detecting to 2.5 micrograms (μg) brain equivalents. Western blot systems using spleenic tissue as the starting material demonstrated detection of PrP^{Sc} in as little as 0.3 mg material following treatment with collagenase. In a recent paper, Safar and co-workers (J. Safar et al., Nature Medicine, Vol 4, No. 10, October 1998) describe a conformation dependent ELISA reportedly able to distinguish various PrP conformers equating to various scrapie strains. The authors claim a sensitivity approximating 10³ IU/ml, within the range of the Western blot assay of this invention.

A different series of studies, Brown and co-workers (ibid) using both spiked and endogenous plasma infectivity models looked at partitioning of model TSEs in several plasma processing steps. In contrast to the method of this invention which monitors for quantity of PrP^{Sc}, Brown et al. monitored actual infectivity. While some of their results are consistent with those found with the method of this invention, the comparison is complicated by the difference in plasma fractionation processes from which the samples were derived and by the fact that a significant amount of input infectivity was not recovered.

In conclusion, the Western blot assay of this invention has been demonstrated to be a robust and sensitive assay for the prion protein. The overall goal of these efforts was to develop an assay that could correlate with TSB infectivity in order to predict infectivity. In hamster brain homogenates, the method is routinely able detect to 5 pg of PrP^{RES} or where infectivity is as low as 10³ IU/ml. This invention provides an inexpensive and rapid assay for the assessment of TSE partitioning in protein purification steps that are used for the production of plasma-derived therapeutics. Previously the rodent bioassay has been used to determine partitioning of infectivity. However, such studies are time consuming and expensive to perform. Immunologically based assays for the detection of PrP^{RES} have been used as a diagnostic marker for TSE infection. These assays rely on the close association of PrP^{Sc} with TSE infectivity.

Western Blot Immunoassay for the Determination of TSE.

Several molecular markers for TSE have been identified. However the most frequently cited are the structural changes observed in the normal (cellular) prion protein (PrP^{C}) which undergoes significant alterations in both secondary and tertiary structures in the diseased state. The ensuing changes have such significant consequences on the physiochemical nature of the PrP molecule, that a novel entity of the PrP^{C} molecule results and is designated PrP^{Sc} (prion protein, scrapie form). The PrP^{Sc} isoform is less soluble in aqueous buffers, and more proteinase resistant than its PrP^{C} counterpart. Treatment of PrP^{Sc} with proteinase-K (PK) results in the removal of only 90 amino acids from the N-terminus. The remaining PrP “core” is denoted PrP^{RES} (prion protein, proteinase resistant). Several studies suggest that the PrP^{Sc} isoform is the infectious agent for TSEs and that it is necessary for disease transmission.

Using the 263K hamster strain of TSE as a model system, the assay is sensitive, specific and linear over a 3-5 log dynamic range. Compared to the rodent bioassay, the assay was shown to detect PrP^{RES} in approximately 10^{3.4} IU/ml which equates to approximately 5 pg of PrP or 8 ng brain equivalents. The Western blot method described herein was used to monitor the partitioning of PrP^{Sc} through three plasma fractionation steps, Cryoprecipitation, Fraction I and Fraction III, that are common to the purification of several plasma-derived therapeutic products including albumin and immunoglobulins. The results from these studies demonstrated a 1 log, 1 log and 4 logs of PrP^{Sc} partitioning away from the effluent fraction. Bioassays for TSE infectivity confirmed the results.

The Western blot immunoassay provided allows for the detection of PrP^{RES} in less than 10 nanograms (ng) of brain tissue or to the limit of approximately 5 picograms (pg) of PrP^{RES}. This level of detection corresponds to the detection of PrP^{RES} in samples where infectivity is not greater than 103 Infectious Units per milliliter (IU/ml). The method utilizes chemiluminescence technology to visualize PrP on the Western blot membranes and requires nominal sample manipulation. The assay is robust and reproducible and may be performed in most laboratories with minimal equipment expenditures. The Western blot assay is able to follow the partitioning of PrP^{Sc} through three protein purification steps that are commonly used for the isolation of several plasma-derived therapeutics.

Materials and Methods

Preparation of brain homogenates: Ten percent scrapie brain homogenates (SBH) were prepared using hamster brains infected with the 263K hamster-adapted agent. Previously frozen hamster brains were allowed to thaw on ice and weighed. Nine volumes of ice-cold Tris buffered saline (TBS) were added and the mixture homogenized using a Polytron apparatus followed by Dounce homogenization using a B pestle. Normal brain homogenates (NBH) were prepared similarly except 25 micromolar (μM) 4-(2-aminoethyl) benzensulfonyl fluoride (AEBSF) was present to inhibit endogenous proteinase activity. Homogenates were stored at −70° C.

Sample Preparation I—Standard Dilution Protocol: A serial dilution on 0.5 log scale was made by mixing 315 microliter (μl) of sample with 684 μl of 0.1 ml bovine serum albumin (BSA) prepared in phosphate buffered saline (PBS) for the samples to be tested. From each, 80 ml was withdrawn and incubated with 350 μg/ml PK for 1 hour at 37° C. To terminate the PK reaction, AEBSF was added to a final concentration of 10 millimolar (mM) and incubated for 10 minutes at ambient temperature. An equal volume of 2×SDS PAGE sample buffer (Novex, San Diego, Calif.) was added and each sample was heated to 100° C. for 10 minutes. In general, 15 ml/well of these solutions were loaded on the gel for analysis. In some cases it was necessary to perform this dilution series in 0.1 ml volumes. For these, the diluents were adjusted accordingly.

Sample Preparation II —Centrifugation Method to Concentrate the PrP signal: During the course of these studies, it was determined that using high speed centrifugation increased the sensitivity of the Western blot assay. To this end, the samples were diluted and treated with PK followed by AEBSF as described above. Following the PK treatment, the vials were centrifuged at 20,000×g for one hour at 4° C. in a fixed angle rotor. The pellet was resuspended in 10 ml of 2×SDS-PAGE sample buffer (Novex), heated for 10 min at 95° C. and the entire sample was electrophoresed.

Polyacrylamide Gel Electrophoresis (PAGE): Samples prepared by either method were electrophoresed on 8.3 cm×7.5 cm, 1.0 mm, 12% SDS-Tris-glycine polyacrylamide gels (Novex CAT# EC60055) for 60 minutes at 125 constant volts.

Western Blot Assay: All volumes described were optimized for the Novex (San Diego, Calif.) gel system using only a single gel or membrane. Improved transfers were obtained when a single gel was blotted per transfer unit. Proportionate increases in working solution volumes are required with additional membranes. Unless otherwise stated, all manipulations were performed at room temperature (22′-25° C.) using molecular biology grade reagents.

Nitrocellulose membranes (0.45 μm, Novex) were soaked in Novex transfer buffer for 5-10 minutes prior to “sandwich” assembly. The transfer from the gel to the nitrocellulose membrane was performed for 60 minutes at 125 milliAmps (mA). Following transfer, the membranes were soaked in TBS (pH 8.0) for 5-10 minutes. The membranes were blocked for 60 minutes in 5% non-fat milk (Organic Valley, CROPP Cooperative, LaFarge, Wis.) dissolved in TBST (TBS with 0.05% Tween) with gentle agitation. Following blocking, the membranes were incubated in a 1:10,000 dilution of 3F4 monoclonal antibody diluted in blocking buffer overnight (12-18 hours) at 4° C. The membranes were rinsed three times with TBST and washed three times for 5 minutes per wash. The membranes were incubated at room temperature for 90 minutes with anti-mouse alkaline phosphatase-conjugated IgG (Catalogue #AM10405, BioSource International or Catalogue #108004, Southern Biotechnologies Associates, Inc.) at a 1:10,000 dilution in 20 ml of 5% blocking buffer. Following incubation in the secondary antibody, the membranes were rinsed with 3 changes of TBST, then washed in TBST for 60 minutes. Alternatively, the membranes can be probed using a directly labeled primary antibody or an antibody fragment, such as the Fab fragment of antibody 3F4 labeled with alkaline phosphatase (see below, optional Method III).

The membranes were agitated for 60 minutes in 50 ml assay buffer (10 mM Tris, 200 MgCl₂, pH 10.0), blotted dry, incubated with 3 ml CDP-Star (Tropix, Bedford, Mass.), containing 0.15 ml NitroBlock II (Tropix, Bedford, Mass.) and were laid on a Whatman 3MM filter paper. The blots were placed in a developing folder (Tropix, Bedford, Mass.) and transferred into a film cassette. The membranes were exposed to film (Kodak XAR-2 or Fuji RX). Generally, exposures of 5, 15, 30, and 60 minutes were obtained, although 90-120 minutes exposures were possible.

Western Blot Quantitation: Unlike previously employed assay, the Western Blot method of this invention has been developed to provide quantitative results. Quantitation of the Western blot films employs an endpoint dilution of a characterized brain homogenate to quantitate infectivity in a solution containing unknown amount of PrP^{Sc} similar to that described for viral assays. This is performed by spiking a known amount of hamster brain homogenate into a given solution that is to be subjected to the manufacturing process. An aliquot is removed prior to performing the manufacturing process and is designated the “prove” sample. The manufacturing step is performed and the resulting fractions are retained for Western blot analysis. Typically two fractions are obtained; a solid, precipitate, fraction and a liquid, effluent fraction. The solid is resuspended in PBS with 0.1% BSA to the same volume as the prove. The prove and/or the liquid fraction is adjusted similarly using the same diluent. The samples are carefully diluted as described above and subjected to Western blot analysis.

For each sample that has been diluted and assayed, a comparison is made to compare the number of detectable lanes. Typically, dilutions are made in 0.5 log increments; therefore, if 10 lanes (Western blot) or wells (ELISA) react positively for PrP^{RES}, the sample is said to have 5 logs of prion detection.

Plasma Fractionation Studies: All plasma fractionations were performed based on the original methods of Cohn. The resulting pellets and effluents were reconstituted to equal volumes for comparison in the Western blot. It has been demonstrated that this assay can be used to monitor the distribution of PrP^{Sc} in samples obtained from three plasma fractionation processes.

Results: This Western blot system has the potential to measure the target protein to a level equivalent of infectivity as low as 10³ IU/ml or approximately 5 pg PrP^{RES} in dilutions derived from infectious brain homogenates (FIG. 1). This level detection corresponds to PrP detection approaching 10 ng of brain tissue equivalents. Specificity of the Western blot was confirmed using a peptide that mimicked the 3F4 epitope on PrP which effectively competed for the PrP signal. The assay was demonstrated to be reproducible by having multiple analysts perform the assay with consistent results.

Optional: Method II: Sometimes, the PrP^{RES} signal was interfered with in certain plasma fractionated samples due to the presence of exogenous proteins. To overcome this impediment, a second protocol (Method II) was developed that employed a high speed centrifugation of the PK-treated dilutions to concentrate the PrP^{RES}. The resulting pellets, enriched for PrP^{RES}, were solubilized with SDS-PAGE sample buffer, and the entire pelleted sample was subjected to Western blot analysis. FIG. 2 shows the enhancement in PrP^{RES} signal in SBH samples following this approach. This treatment increased the sensitivity of PrP^{RES} detection by approximately 1.5 logs and after development was used in all subsequent fractionation steps studied and is the preferred mode of practicing the invention.

The detection of PrP^{RES} by Western blot was compared to infectivity using the rodent bioassay. A sample of SBH was serially diluted in 0.5 log increments using both standard and centrifuged dilution protocols and analyzed using the Western blot. Aliquots only from the standard dilution protocol were subjected to the bioassay (Table I). The undiluted 10% SBH used in this study was determined to have a titer of 10^{7.9} IU/ml by the rodent bioassay. The Western blot detected PrP^{RES} immunoreactivity equating to 10^{4.4} IU/ml infectivity in samples derived from the standard sampling procedure. The centrifugation protocol allowed detection of PrP^{RES} in as few as 10^{3.4} IU/ml of sample homogenate. Since performing this specific experiment, further progress has allowed us to improve PrP^{RES} detection in as little as 10³ IU/ml.

To address the issue of diluents and their impact on the dilution properties of PrP^{Sc}, several dilution mediums were tested including PBS, TBST, sarkosyl, human plasma, BSA and hamster NBH. The sensitivity of the Western blot assay was dependent on the diluent used for sample preparation. Of all the diluents tested, BSA, was the most effective at maintaining linearity of dilution, while still retaining the greatest sensitivity.

From about 0.5 to about 1 log differences in PrP^{RES} levels can be readily discerned with the Western blot assay. Application of the Western blot to the measurement of PrP^{Sc} disposition through plasma processing can be quantitated and compared to data obtained from bioassay analysis.

Optional: Method III: Immunoassay detection of prion protein (PrP^{RES}) can be obscured by non-specific interactions from other proteins, particularly immunoglobulin fragments that may be present in biological samples. In some embodiments, the present invention provides chemical and enzymatic methods to produce an antibody-enzyme conjugate that minimizes the non-specific signal. The use of this conjugate leads to a highly specific method for the detection of prion protein.

Results from Western blot assay can sometimes be obscured by non-specific signals from immunoglobulin-rich samples, particularly intermediate samples obtained for analysis from an immunoglobulin preparation process. Often, these non-specific signals have an apparent molecular weight of 20 to 40 kD, similar to the prion protein (See FIG. 4). These signals can be reduced by protein G treatment, which removes immunoglobulins, indicating that immunoglobulins are the signal source.

It is possible that the non-specific signals are immunoglobulin fragments degraded by proteinase K (PK) digestion, a treatment allowing differentiation between pathogenic PrP^{Sc} and normal PrP^{C}. Indeed, these signals shifted to the molecular weight of immunoglobulin, or 140 kDa, when the PK treatment step was omitted (FIG. 5). Furthermore, when primary antibody was omitted from the system, the non-specific signals remained (FIG. 5, left panel). This result indicated that these non-specific signals were the result of interactions between immunoglobulin fragments and the secondary antibody. Further experiments indicated that the antibody Fc portion appeared to be responsible for these non-specific interactions.

To eliminate the secondary antibody from the assay system, anti-prion antibody 3F4-Fab fragment was generated and directly conjugated to alkaline phosphatase to produce a detection probe binding directly to prion protein, 3F4-Fab-AP (See the schematic diagram presented in FIG. 6). The anti-PrP monoclonal antibody 3F4 was purified using a peptide affinity column and fragmented using immobilized pepsin to produce F(ab′)2, where the Fc fragment was removed. Subsequently, Fab fragments with free sulfhydryl groups were produced using the reducing agent mercaptoethylamine (MEA). Alkaline phosphatase was activated using the cross-linker sulfosuccinimidyl maleimidomethyl cyclohexane carboxylate (SMCC, a heterobifunctional linker capable of linking compounds with amine and sulfhydryl groups). Both reagents were then purified. Finally, reduced Fab and activated alkaline phosphatase were mixed to produce 3F4-Fab-AP, and the product was purified using size-exclusion chromatography. The 3F4-Fab-AP was tested in a direct Western blot assay and compared to the two-step Western blot (with labeled secondary antibody) using PK-treated PrP^{Sc} and immunoglobulin samples (See FIG. 7).

In summary, the non-specific signals were shown to result from interactions between immunoglobulin fragments and the Fc portion of secondary antibody used in the two-step Western blot assay system. Using directly labeled 3F4-Fab-AP resulted in approximately 100-fold or more reduction in non-specific signal for analyses of immunoglobulin-rich samples (FIG. 7, right panels). The sensitivity of the direct Western was similar to the two-step format (FIG. 7, left panels).

### BRIEF DESCRIPTION OF THE FIGURES

FIG. 1. Demonstration of sensitivity of assay system using the Western blot protocol. Hamster SBH was diluted in 0.1% BSA/PBS in 0.5 log increments, treated with PK and subjected to Western blot analysis as described in the Materials and Methods. The numbers below the panel illustrate the relative amounts of putative infectivity in the corresponding dilution (IU/ml) or as related to the volume loaded on the gel (IU/lane).

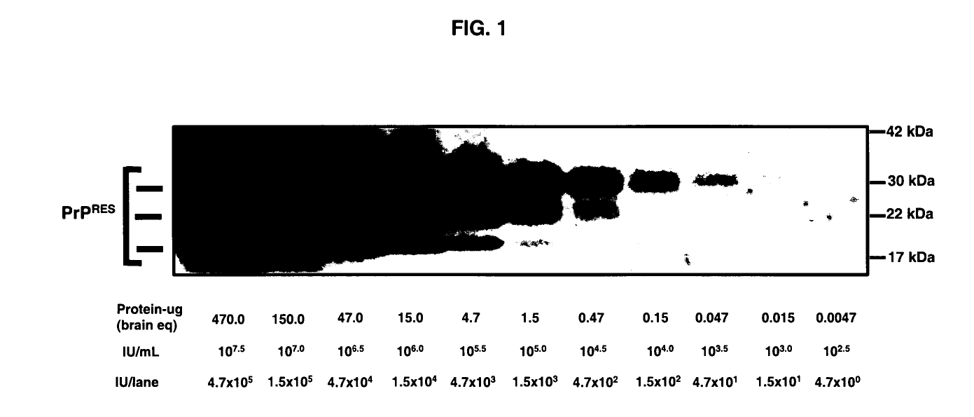

FIG. 2. Centrifugation concentrates the PrP^{Sc}signal and increases the number of lanes detected in Western blots. (A) 0.5 log dilutions of SBH generated using standard dilution methods as described in Materials and Methods. (B) The remaining dilutions generated for (A) were concentrated by centrifugation. The resulting pellets were subjected to Western blot analysis and demonstrate an increase in the level of detection.

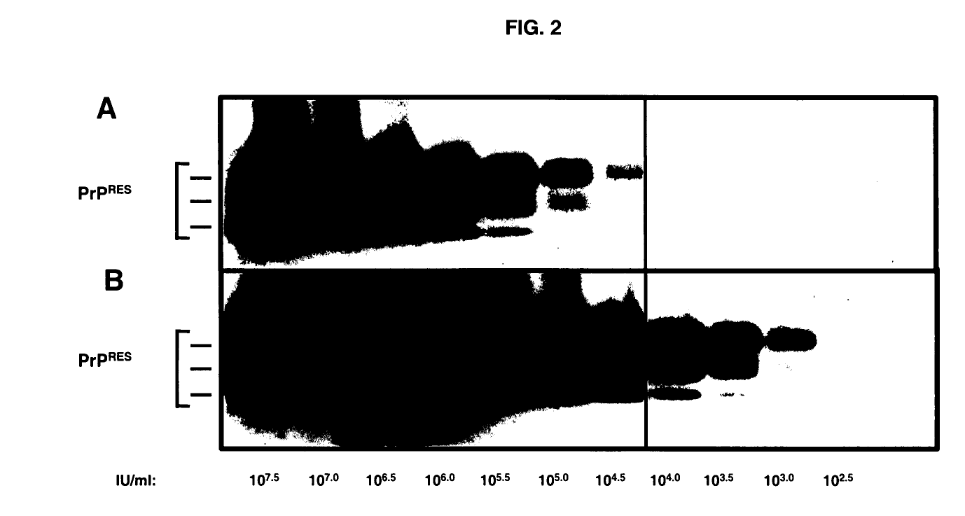

FIG. 3. Application of the Western blot to the analysis of the plasma processing steps. SBH spiked Cryoprecipitate (A), Fraction I (B) and Fraction III (C) steps generated Prove, Effluent and Paste samples which were subjected to Western blot analysis.

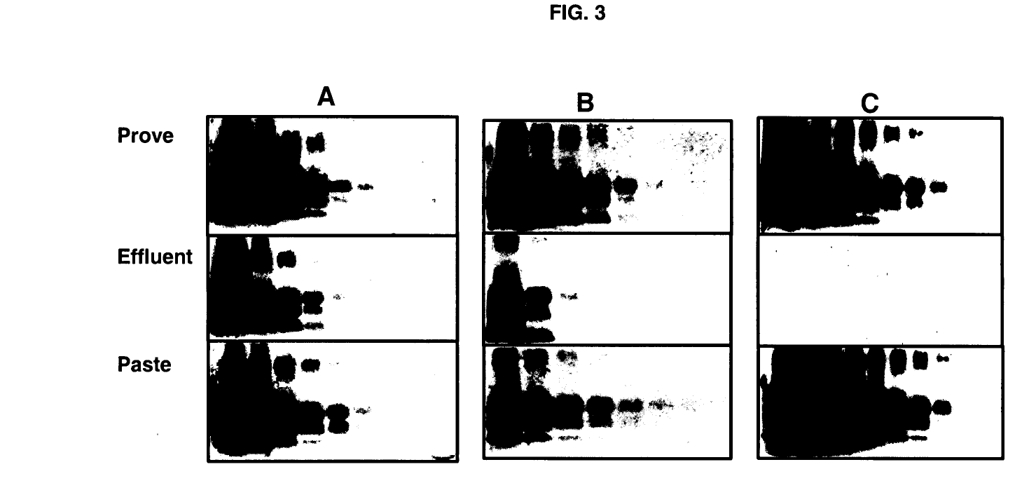

FIG. 4. Non-specific signal from immunoglobulin-rich samples may obscure the two-step Western blot result. PrP^{Sc} or immunoglobulin samples were treated with PK, diluted in half-log series, and analyzed using the two-step Western blot assay. The non-specific signal from immunoglobulin (right panel) resembles the real prion signal (left panel).

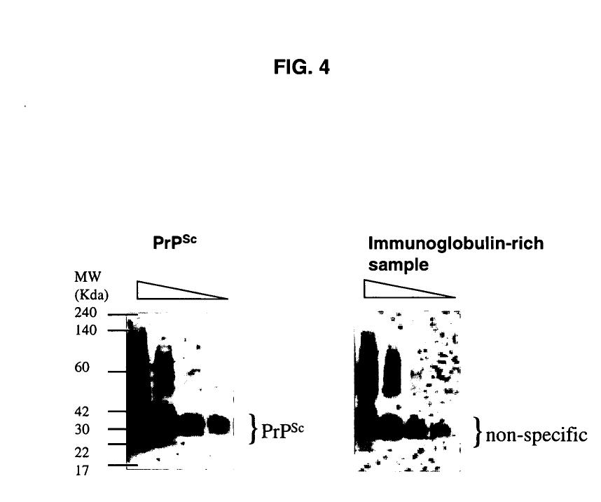

FIG. 5. Interaction between secondary antibody and immunoglobulin in the sample can lead to non-specific signal. A mixture of PrP and immunoglobulin was analyzed by using the two-step Western blot in the absence of PK treatment. The non-specific signal shifted to ˜140 kDa (left and right panel), revealing its immunoglobulin origin (middle panel). The primary Ab was omitted in the left panel.

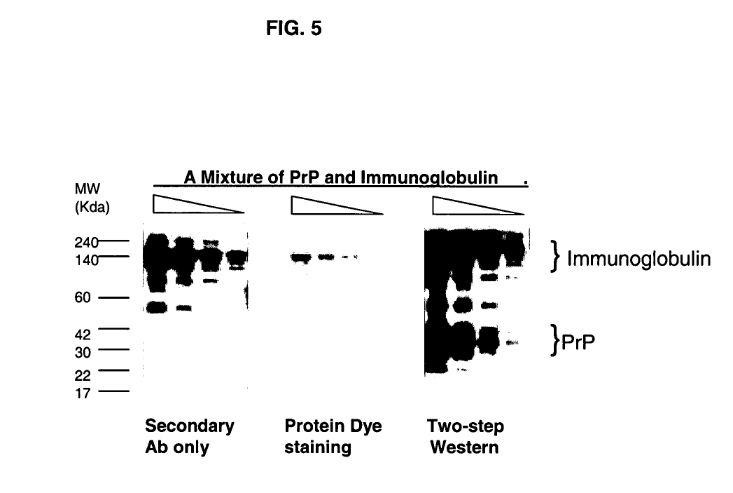

FIG. 6. Schematic representation of the production of 3F4-Fab-AP (on the production of antibody fragment Fab, and the production of antibody fragment Fab-enzyme conjugate of the invention).

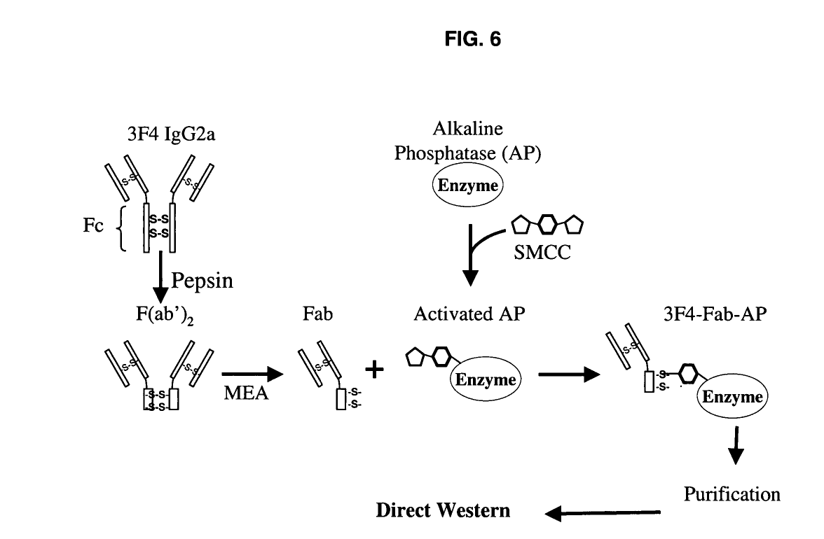

FIG. 7. Comparison of direct and two-step Western blot procedures. 10% SBH containing PrP^{Sc} or immunoglobulin samples were diluted at 1:500, treated with PK, mixed 1:1 with SDS sample buffer, and diluted in 0.5-logs. The same protocol was used for the direct Western as for the two-step procedure, except that a 1:5000 dilution of 3F4-Fab-AP was used in the direct method.

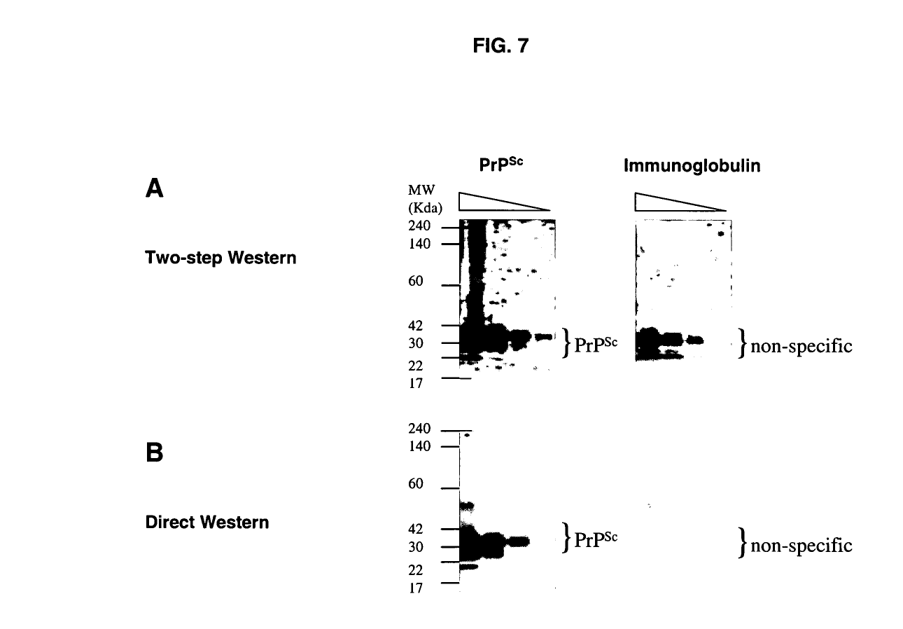

TABLE I
Direct comparison of Western blot data with bioassay. Samples of SBH
were diluted in 0.5 log increments. Each dilution was analyzed using the
Western blot assay, while every other dilution was analyzed in the rodent
bioassay. Analysis of the data derived from the bioassay demonstrates that
the infectivity titer of the undiluted SBH to be 10^{7.9} IU/ml. Western blot
lanes positive for PrP^{RES} signal are designated with a plus (+) sign,
negative lanes are designated with a minus (−) sign.

| | Western blot | | Bioassay Results | | |
|---|---|---|---|---|---|
| | Standard | Centrifuged | | Incubation | |
| SBH Dilution | Method | Method | Dead/total | (days) | IU/ml |
| 0 | + | + | ND | ND | 10^{7.9} |
| −0.5 | + | + | ND | ND | 10^{7.4} |
| −1.0 | + | + | 5/5 | 72 ± 2 | 10^{6.9} |
| −1.5 | + | + | ND | ND | 10^{6.4} |
| −2.0 | + | + | 5/5 | 80 ± 2 | 10^{5.9} |
| −2.5 | + | + | ND | ND | 10^{5.4} |
| −3.0 | + | + | 5/5 | 86 ± 2 | 10^{4.9} |
| −3.5 | + | + | ND | ND | 10^{4.4} |
| −4.0 | − | + | 5/5 | 91 ± 1 | 10^{3.9} |
| −4.5 | − | + | ND | ND | 10^{3.4} |
| −5.0 | − | − | 4/5 | 97 ± 0 | 10^{2.9} |
| −5.5 | ND | − | ND | ND | 10^{2.4} |
| −6.0 | ND | ND | 5/5 | 117 ± 6 | 10^{1.9} |
| −6.5 | ND | ND | ND | ND | 10^{1.4} |
| −7.0 | ND | ND | 1/5 | 125 | 10^{0.9} |
| −7.5 | ND | ND | ND | ND | 10^{0.4} |
| −8.0 | ND | ND | 0/5 | 0/5 | − |

ND, not determined

1. An immunoassay method useful for determining the presence of TSE protein in a biological sample, comprising the steps of:
a) preparing a relevant biological sample for a Western blot immunoassay;
b) performing the Western blot immunoassay on such sample for TSE protein; and
c) quantitating the TSE protein present wherein the immunoassay is carried out using a labeled TSE-specific antibody or antibody fragment.

2. The method of claim 1, wherein the labeled TSE-specific antibody or antibody fragment is labeled by covalent linkage to an enzyme.

3. The method of claim 2, wherein the enzyme is selected from the group consisting of alkaline phosphatase, horseradish peroxidase, amylate dehydrogenase, staphylococcal nuclease, δ-5-steroidisomerase, yeast alcohol dehydrogenase, α-glycerophosphate dehydrogenase, triose phosphate isomerase, ribonuclease, urease, catalase, glucose-6-phosphate dehydrogenase, glucoamylase, β-galactosidase, acetylcholinesterase, asparaginase, and glucose oxidase.

4. The method of claim 2, wherein the enzyme is alkaline phosphatase.

5. The method of claim 2, wherein the covalent linkage is accomplished by activation of alkaline phosphatase using sulfosuccinimidyl maleimidomethyl cyclohexane carboxylate.

6. The method of claim 1, wherein the TSE-specific antibody is 3F4 monoclonal antibody or antibody fragment.

7. The method of claim 1 wherein the biological sample is chosen from the group consisting of a homogenized tissue, plasma, and a plasma processing fraction sample.

8. The method of claim 1 wherein the biological sample is prepared by:
a) diluting with a physiologically compatible buffer to form a buffered dilution; and
b) treating the buffered dilution with proteinase-K to form a buffered proteinase-K dilution.

9. The method of claim 8, wherein the biological sample is diluted serially, up to nine logs.

10. The method of claim 8, wherein the physiologically compatible buffer is a buffered saline solution.

11. The method of claim 10, wherein the buffer component of the buffered saline solution is chosen from the group consisting of PBS, 0.1% BSA in PBS, and Tris buffered saline.

12. The method of claim 8, wherein an aliquot of the diluted sample is treated with proteinase-K.

13. The method of claim 12, wherein the proteinase-K treated dilution is concentrated.

14. The method of claim 13, wherein the proteinase-K treated dilution is concentrated by centrifugation or filtration.

15. The method of claim 14, wherein the concentrated proteinase-K treated solution is resuspended and subjected to Western blot immunoassay, comprising the steps of:
a) separating the proteinase-K treated samples electrophoretically;
b) transferring the separated samples to a membrane;
c) adding a blocking agent to the membrane containing the separated samples;
d) incubating the membranes with a labeled antibody or antibody fragment capable of binding to TSE protein;
e) washing the incubated membrane with a low salt buffer to remove any non-binding antibodies and proteins;
f) measuring the signal produced by counting the number of lanes with a detectable signal.

16. The method of claim 15, wherein the Western blot immunoassay utilizes the monoclonal antibody, 3F4, or a fragment thereof as the labeled antibody or antibody fragment for the identification of the TSE protein on the membrane.

17. The method of claim 15, wherein the results of the Western blot are quantified by:
a) determining the number of immunoreactive lanes on the membrane that contain a signal attributable to the presence of pathogen protein; and
b) approximating the amount of pathogen protein present to the limits of detection.

18. An immunoassay method useful for determining the presence of TSE protein in a biological sample, comprising the steps of:
a) diluting the biological sample with a physiologically compatible buffer to form a buffered dilution;
b) treating the buffered dilution with proteinase-K to form a buffered proteinase-K dilution;
c) performing a Western blot immunoassay for TSE protein on the biological sample; and
d) quantitating the TSE protein present, wherein the immunoassay is carried out using a labeled TSE-specific antibody or antibody fragment.

19. A quantitative Western blot immunoassay method useful for the determination of TSE protein clearance during the processing of plasma products, comprising the steps of:
a) preparing an aliquot of a relevant first sample chosen from plasma or a processed plasma sample for a Western blot immunoassay;
b) performing the Western blot assay for TSE protein on such first sample;
c) quantitating TSE protein in the first sample aliquot;
d) subjecting the first plasma sample or first processed plasma sample to a processing treatment producing a second sample;
e) performing a Western blot assay for TSE protein on an aliquot of the second sample;
f) quantitating TSE protein results in the second sample; and
g) comparing the quantitative TSE protein in the first sample to the quantitative TSE protein in the second sample to determine if the processing step provides detectable TSE protein clearance,
wherein the Western blot assays are carried out using a labeled TSE-specific antibody or antibody fragment.

20. A method of determining TSE protein clearance by a particular plasma processing step:
a) taking a sample of plasma paste prior to the processing step of interest;
performing a Western blot analysis on the plasma paste sample to quantitate TSE protein content;
b) spiking the plasma paste with known amount of scrapie brain homogenate; resuspending the spiked paste;
c) taking an aliquot of the resuspended spiked paste; and performing a Western blot analysis on the resuspended spiked paste to quantitate TSE protein content;
d) performing the processing step of interest on the plasma paste thereby obtaining a processed paste and an effluent;
e) taking a sample of the processed paste and performing a Western blot analysis to quantitate TSE protein content in the processed paste; and
f) comparing TSE protein content of the samples tested to determine if satisfactory TSE protein clearance has been obtained,
wherein the Western blot assays are carried out using a labeled TSE-specific antibody or antibody fragment.

21. An immunoassay method useful for determining the presence of TSE protein in a biological sample, comprising the steps of:
a) preparing a relevant biological sample for a Western blot immunoassay;
b) performing the Western blot immunoassay on such sample for TSE protein; and
c) quantitating the TSE protein present
wherein the immunoassay is carried out using a labeled TSE-specific antibody fragment lacking the Fc portion of the antibody.

22. The immunoassay of claim 21, wherein the TSE-specific antibody fragment is the Fab portion of antibody 3F4.
